(19) United States
(12) Patent Application Publication
(10) Pub. No.: US 2013/0066278 A1
(43) Pub. Date: Mar. 14, 2013

(54) MODULAR VALVE APPARATUS

(71) Applicants: James B. Holland, Clinton, NC (US); George Hsu, Tiachung-City (TW)

(72) Inventors: James B. Holland, Clinton, NC (US); George Hsu, Tiachung-City (TW)

(73) Assignee: KMQ, INC., Kenansville, NC (US)

(21) Appl. No.: 13/599,174

(22) Filed: Aug. 30, 2012

Related U.S. Application Data
(60) Provisional application No. 61/533,921, filed on Sep. 13, 2011.

(51) Int. Cl. F16K 15/04; A61M 5/31

(52) U.S. Cl. 604/186; 137/539; 604/207

(57) ABSTRACT

Various embodiments are directed to a modular valve apparatus. The modular valve apparatus includes a retaining component having an opening on either end. The modular valve apparatus further includes a cap component having a hole bored through. The cap component may be adapted to be coupled with the retaining component. A generally annular spring component may be disposed within the retaining component and seated on a lower end extending axially toward an upper end of the retaining component. A check ball may be disposed within the retaining component and seated on the spring component. The check ball may be adapted to block the opening bored through the cap component while being held in place by the spring component.

### CROSS-REFERENCE TO RELATED APPLICATIONS

This application claims priority to and the benefit of U.S. Provisional Application Ser. No. 61/533,921 filed Sep. 13, 2011 and entitled, “Modular Valve Apparatus”.

### BACKGROUND

Valve assemblies are commonplace in syringe assemblies used in the veterinary industry. Such syringe assemblies may be used to inject or inoculate livestock as necessary. Many of these syringe assemblies are reusable and utilize relatively large amounts of fluid that need to be regulated from source through needle tip to ensure a proper dosage is given during an injection. Sometimes the fluid comes from a reservoir and is delivered to a syringe assembly through a hose that is coupled to an input port. Sometimes the fluid is stored in a disposable/replaceable bottle that is mountable to the syringe gun itself. In either case, fluid is typically drawn from its source into a barrel chamber and out a needle tip. Along the way multiple valve assemblies may be used to help regulate the flow of the fluid through the larger syringe assembly.

Inserting the valve assemblies into the syringe assemblies can be difficult since the valve assemblies are comprised of multiple components. If the components are not inserted properly the valve assembly may malfunction causing the entire syringe assembly to malfunction.

Simplifying the valve assembly may reduce the time required to assemble the syringe assembly and reduce the potential of errors during assembly.

### BRIEF DESCRIPTION OF THE DRAWINGS

FIG. 1 illustrates one embodiment of a modular valve apparatus showing various components in an exploded view.

FIG. 2 illustrates one embodiment of a cap component of a modular valve apparatus.

FIG. 3 illustrates one embodiment of a spring component of a modular valve apparatus.

FIG. 4 illustrates one embodiment of a retainer component of a modular valve apparatus.

FIG. 5 illustrates one embodiment of an assembled modular valve apparatus.

FIG. 6 illustrates one embodiment of an assembled modular valve apparatus to be coupled between a nib component and a barrel component of a syringe assembly.

FIG. 8 illustrates one embodiment of two assembled modular valve apparatus implemented in a bottle mount syringe assembly.

### DETAILED DESCRIPTION

In various embodiments, a modular valve apparatus may address common deficiencies associated with regulating the flow of a fluid to be dispensed in a syringe assembly. The modular valve apparatus may combine a spring and a check ball within a cage-like retainer. Current implementations of valve assemblies within a larger syringe assembly make assembly difficult because the check ball and spring may be handled and installed separately into the fluid path of the syringe assembly. The chance that either the ball or spring is incorrectly inserted exists. In creating a sealable cage-like retainer for the ball and spring, the chance of incorrectly inserting the spring or ball is eliminated.

Reference is now made to the drawings, wherein like reference numerals are used to refer to like elements throughout. In the following description, for purposes of explanation, numerous specific details are set forth in order to provide a thorough understanding thereof. It may be evident, however, that the novel embodiments can be practiced without these specific details. In other instances, well known structures and devices are shown in block diagram form in order to facilitate a description thereof. The intention is to cover all modifications, equivalents, and alternatives falling within the spirit and scope of the claimed subject matter.

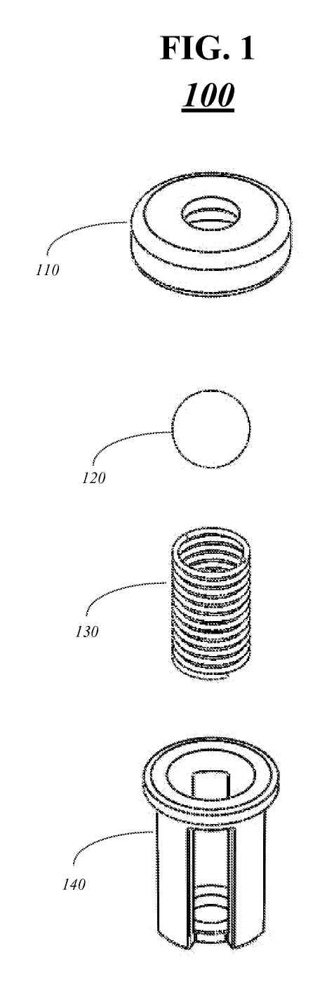

FIG. 1 illustrates one embodiment of a modular valve apparatus 100 showing various components in an exploded view including a cap component 110, a check ball 120, an annular spring component 130, and a retainer component 140. The cap component 110 may be adapted to couple with the retaining component 140 such that check ball 120 and spring 130 are securely contained within the confines of the retaining component 140 when coupled together. When assembled in this manner the cap component 110, check ball 120, spring 130, and retaining component 140 form a modular valve apparatus 100.

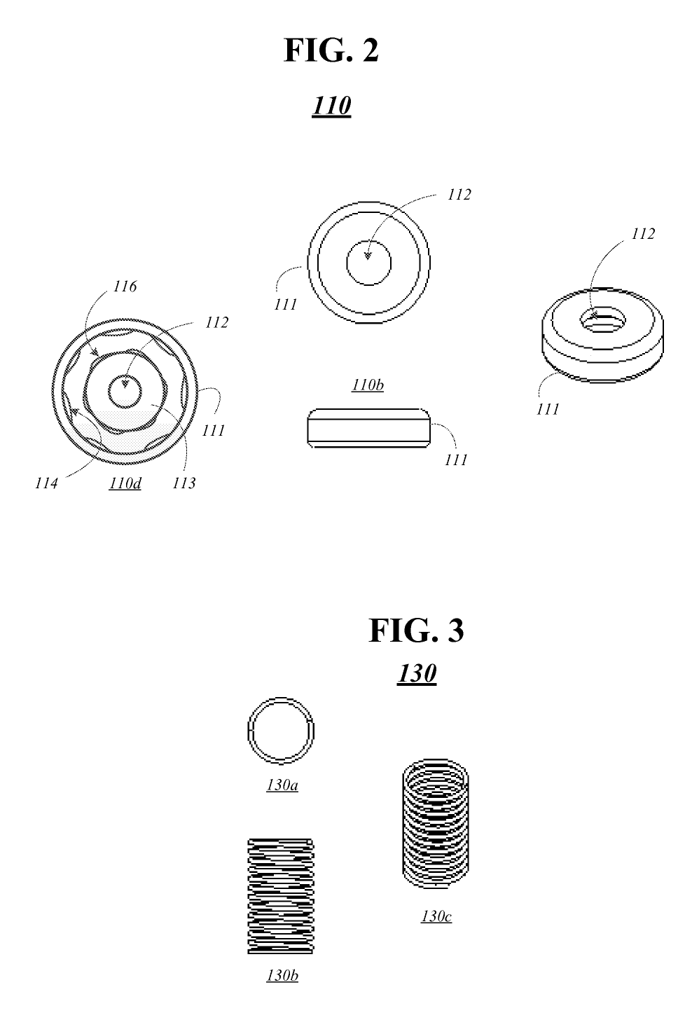

FIG. 2 illustrates one embodiment of a cap component 110 for the modular valve apparatus 100. The cap component 110 is shown in a top view 110a, a side view 110b, a perspective view 110c, and a bottom view 110d. In general, the cap component 110 may include an opening 112 that allows fluid to flow through when the check ball 120 is not blocking the opening 112. In some embodiments, the cap component 110 may be generally cylindrical in shape and include a circular opening 112 through its center axis that allows fluid to flow through when the check ball 120 is not blocking the opening 112. The cap component 110 may be threaded such that it can be screwed to the retaining component 140 which may be reciprocally threaded. For example, in the bottom view 110d, the cap component 110 may include outer threads 114 positioned on the inner portion of an outer rim 111 of the cap component 110. The cap component 110 may also include inner threads 116 positioned on the outer portion of an inner rim 113 of the cap component 110. The inner threads 116 and outer threads 114 may be adapted to couple with reciprocal threads on the retaining component 140.

FIG. 3 illustrates one embodiment of a spring component 130 of a modular valve apparatus 100. The spring component 130 is shown in a top view 130a, a side view 130b, and a perspective view 130c. The spring component 130 may be generally annular along a vertical axis and have a diameter slightly smaller than that of the check ball 120. This allows the check ball 120 to be seated upon spring 130 when confined within retaining component 140. The spring component 130 may be made of metal, plastic or other suitable material. The spring component 130 may be designed to completely restrict the flow of fluid in an inactive state. For instance, the spring component 130 is gauged to exert enough force to hold the check ball 120 against the opening 112 in cap component 110 when confined and seated within the retaining component 140 with no other forces acting upon the spring component 130. In this state, the check ball 120 completely blocks opening 112 and no fluid can enter the through the opening 112 into the cap component 110. When an opposing force greater than that exerted by the spring component 130 is applied against the check ball 120, the spring component 130 may be compressed. In this active state, the check ball 120 no longer blocks opening 112 thereby allowing fluid to flow through opening 112. It is typically the pressure applied to the fluid that overcomes the force of the spring component 130.

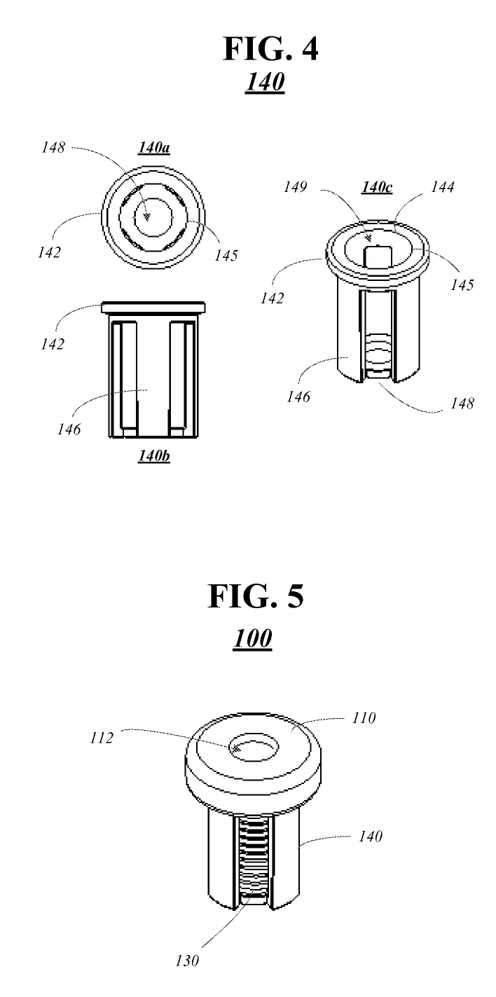

FIG. 4 illustrates one embodiment of a retainer component 140 of a modular valve apparatus 100. The retaining component 130 is shown in a top view 140a, a side view 140b, and a perspective view 140c. In one embodiment, the retaining component 140 may be generally cylindrical and tapered having an opening on both the smaller diameter end 148 and the larger diameter end 149. The retaining component 140 may be comprised of molded plastic, metal or other suitable material. On the larger opening end 149, the retaining component 140 may be threaded on both its outer surface 142 and also threaded on the inner surface of the opening 144 such that corresponding threads in the cap component 110 may cooperatively engage the retaining component 140 and seal the spring component 130 and check ball 120 within the retaining component 140. An O-ring 145 may be seated atop the larger opening end 149 to provide an air and liquid tight seal between cap component 110 and retaining component 140. The O-ring 145 may be made from rubber, plastic or other suitable material.

In one embodiment, the retaining component 145 may be comprised of rib-like structures 146 that provide structural integrity for the modular valve apparatus 100. There may be multiple rib-like structures 146 arranged to form a cage. The space between each rib-like structure 146 is determined so as to allow neither the spring component 130 nor the check ball 120 to escape the cage. The spaces between each rib-like structure 146 further allow fluid to flow through the modular valve apparatus 100 when the check ball 120 is unseated from its position against opening 112. The base of the retaining component 140 may include an opening 148 but also includes a rim adapted to seat the spring component 130 and retain it in an operative position with the check ball 120. Thus, the diameter of the spring component 130 exceeds that of the opening 148 at the base of the retaining component 140.

The retaining component 140 need not be tapered in design. The retaining component 140 may have similar sized openings on either end so long as the design permits the spring component 130 and check ball 120 to be securely seated within the retaining component 140 and further allows fluid to flow unrestricted through the modular valve apparatus 100 when the check ball 120 is unseated from its position against opening 112.

FIG. 5 illustrates one embodiment of an assembled modular valve apparatus 100. In this illustration, the cap component 110 has been screwed onto the retaining component 140 using the threaded characteristics of both the retaining component 140 and the cap component 110. The O-ring 145 (not seen) is slightly compressed between the cap component 110 and the top of the retaining component 145 in this coupling to provide a fluid tight seal. Prior to coupling the cap component 110 with the retaining component 140, the spring component 130 and the check ball 120 were placed within the retaining component 140. The spring component 130 may be seated on the bottom rim on the interior of the retaining component 140. The spring component 130 may extend upward toward the larger opening end 149 of the retaining component 140. While not visible in this figure, the check ball 120 may sit atop the spring component 130 and may be forced upward by the tension in the spring component 130. This tension causes the check ball 120 to be held against the cap component 110 firmly blocking the opening 112 of the cap component 110. When a superior opposing force on fluid is applied against the check ball 120, the spring component 130 may compress causing the check ball 120 to become displaced from opening 112 and allow fluid to flow through the modular valve apparatus 100.

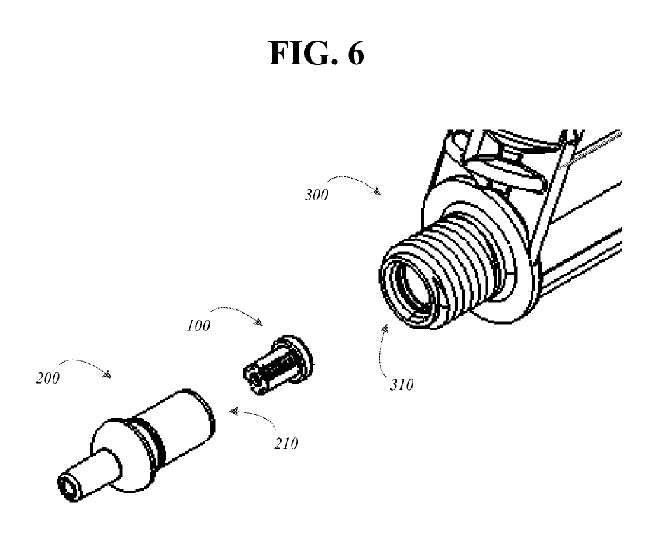

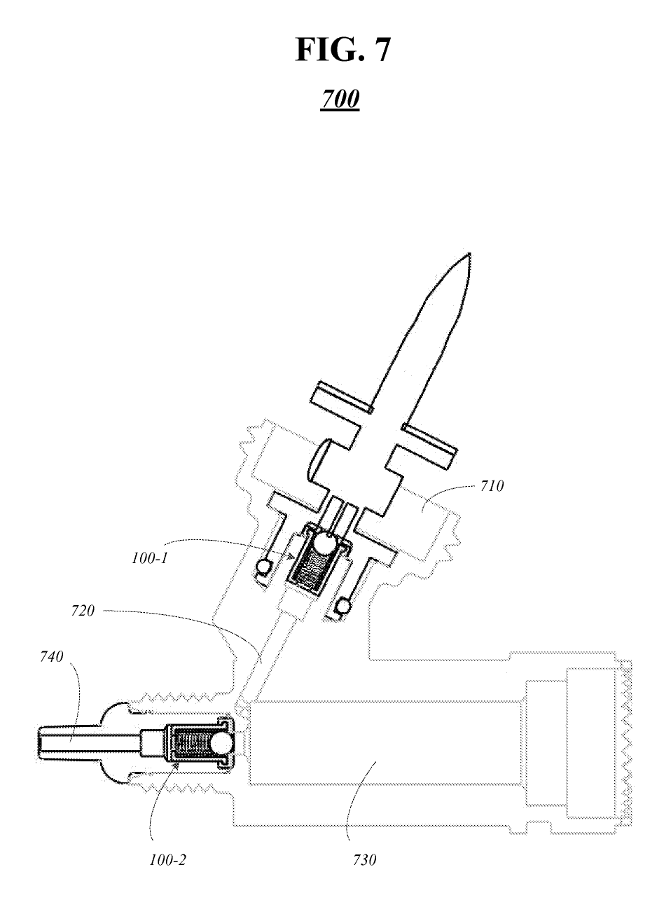

FIG. 6 illustrates one embodiment of an assembled modular valve apparatus 100 to be coupled between a nib component and a barrel component of a syringe assembly. The syringe assembly is only partially shown here illustrating a bottle mount and barrel portion 300 as well as nib portion 200 that leads to a needle assembly. The modular valve assembly 100 is adapted to fit within the nib portion 200 via opening 210. The entire nib portion 200 may then be inserted into the barrel portion 300 via opening 310. The modular valve apparatus 100 completely blocks, in a fluid tight manner, the open space between the barrel portion 300 and the nib portion 200 when the barrel portion 300 is coupled with the nib portion 200. Thus, no fluid can flow from the barrel portion 300 through the nib portion 200 and ultimately out a needle portion without the cooperation of the modular valve apparatus 100. Fluid may only flow through the modular valve apparatus 100 when a pressure on the fluid in the barrel portion 300 overcomes the tension in the spring component 130 of the modular valve apparatus 100. The embodiments are not limited to this example. For instance, the modular valve apparatus may be used at several points within the syringe assembly to regulate the flow of fluid.

FIG. 7 illustrates one embodiment of two assembled modular valve apparatuses 100-1, 100-2 implemented in a bottle mount syringe assembly 700. The first modular valve assembly 100-1 may be positioned at a coupling point between a bottle mount 710 and a draw tube 720 that leads to a barrel chamber 730. In operation, a plunger/piston mechanism (not shown) is pulled away from the needle end of the syringe assembly 700 causing vacuum pressure to pull at the check ball 120 in modular valve assembly 100-1. When the force is sufficient to compress spring mechanism, the check ball will move away from opening in the cap component allowing fluid to flow from the bottle through the draw tube 720 and into the barrel chamber 730. Once the chamber is full or has a desired amount of fluid the piston mechanism is released and the pressure on the check ball 120 in modular valve assembly 100-1 subsides allowing the spring component 130 to re-seat the check ball 120 against the opening 112 in the cap component 110 effectively closing off access to the bottle.

The second modular valve assembly 100-2 may be used in dispensing the fluid out of the barrel chamber 730 and into the nib portion 740 of the syringe assembly. The nib portion 740 ultimately connects with a needle portion (not shown). To dispense fluid, the plunger/piston (not shown) is forced down the barrel compressing the fluid through the opening 112 of the cap component 110 of the modular valve assembly 110-2 and against the check ball 120. When pressure from the compressed fluid reaches a sufficient level, the check ball 120 will compress the spring component 130 and fluid will be allowed to flow through the opening 112 and around the check ball 120 into and about the chamber of the retaining component 140. So long as sufficient pressure on the plunger/piston mechanism is maintained fluid will flow out the other end 148 of the retaining component 140 and into the nib portion 740. When the pressure on the plunger/piston mechanism is released the spring component 130 will compress the check ball 120 back against the opening 112 of the cap component 110 such that no fluid can flow through the opening 112 of the cap component 110 in modular valve assembly 110-2.

It should be noted that the modular valve assembly 100 is a one-way valve in that fluid may only flow in one direction and cannot flow in the reverse direction through the modular valve assembly 100.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A modular valve apparatus comprising:
a retaining component having an opening on an upper end and a lower end;
a cap component having a hole bored through, the cap component adapted to be coupled with the retaining component;
an annular spring component adapted to be disposed within the retaining component, the spring component seated at a lower end of the retaining component extending toward an upper end of the retaining component; and
a check ball disposed within the retaining component and seated on the spring component, the check ball adapted to block the hole bored through the cap component by tension in the spring component.

2. The modular valve apparatus of claim 1, the retaining component being generally cylindrical and tapered having an opening on both the smaller diameter end and the larger diameter end.

3. The modular valve apparatus of claim 1, the retaining component comprised of multiple rib-like structures extending from the lower end to the upper end, each rib-like structure spaced apart to allow fluid to pass through.

4. The modular valve apparatus of claim 3, each rib-like structure spaced close enough to prevent the check ball and the spring component to pass through.

5. The modular valve apparatus of claim 1, the cap component being generally cylindrical with the hole bored through its center axis.

6. The modular valve apparatus of claim 1, the retaining component comprised of plastic, metal, or a combination thereof.

7. A syringe assembly comprising:
a barrel portion;
a nib portion; and
a modular valve apparatus disposed between the barrel portion and the nib portion, the modular valve apparatus comprising:
a retaining component having an opening on an upper end and a lower end;
a cap component having a hole bored through, the cap component adapted to be coupled with the retaining component;
an annular spring component adapted to be disposed within the retaining component, the spring component seated at a lower end of the retaining component extending toward an upper end of the retaining component; and
a check ball disposed within the retaining component and seated on the spring component, the check ball adapted to block the hole bored through the cap component by tension in the spring component,
wherein fluid from the barrel portion must flow through the modular valve apparatus to reach the nib portion.

8. A syringe assembly comprising:
a barrel portion;
a bottle mount; and
a modular valve apparatus disposed between the barrel portion and the nib portion, the modular valve apparatus comprising:
a retaining component having an opening on an upper end and a lower end;
a cap component having a hole bored through, the cap component adapted to be coupled with the retaining component;
an annular spring component adapted to be disposed within the retaining component, the spring component seated at a lower end of the retaining component extending toward an upper end of the retaining component; and
a check ball disposed within the retaining component and seated on the spring component, the check ball adapted to block the hole bored through the cap component by tension in the spring component,
wherein fluid from the bottle mount portion must flow through the modular valve apparatus to reach the barrel portion.
